(19) United States
(12) Patent Application Publication
(10) Pub. No.: US 2025/0170154 A1
(43) Pub. Date: May 29, 2025

(54) Genistein phosphate derivatives

(71) Applicant: GreenStone Biosciences, Inc., Palo Alto, CA (US)

(72) Inventors: Joseph C Wu, Palo Alto, CA (US); Syed Mukhtar Ahmed, Milpitas, CA (US); Rabindra Vishwadev Shivnaraine, Palo Alto, CA (US); Honit Piplani, Sunnyvale, CA (US); Paul D Pang, San Jose, CA (US)

(73) Assignee: GreenStone Biosciences, Inc., Palo Alto, CA (US)

(21) Appl. No.: 18/955,706

(22) Filed: Nov. 21, 2024

Related U.S. Application Data
(60) Provisional application No. 63/603,118, filed on Nov. 28, 2023.

(51) Int. Cl. A61K 31/665; C07F 9/655

(52) U.S. Cl. CPC A61K 31/665; C07F 9/65522

(57) ABSTRACT

New genistein phosphate derivatives, pharmaceutical compositions containing them, and their medicinal uses.

### CROSS-REFERENCE TO RELATED APPLICATIONS

This application claims the benefit under 35 USC 119(e) of U.S. Application No. 63/603,118, filed 28 Nov. 2023 and entitled “Genistein phosphate derivatives”, the entire content of which is incorporated into this application by reference.

### FIELD OF THE INVENTION

This invention relates to new genistein phosphate derivatives, pharmaceutical compositions containing them, and their medicinal uses.

### DESCRIPTION OF THE RELATED ART

### Genistein

Genistein, 5,7-dihydroxy-3-(4-hydroxyphenyl)-4H-chromen-4-one; 4′,5,7-trihydroxyisoflavone; sometimes incorrectly 4′,5,7-trihydroxyflavone, is a phytoestrogen belonging to the class of soy isoflavones. It was first isolated in 1899 from the dyer's broom, Genista tinctoria; its structure was established in 1926; and it was chemically synthesized in 1928 (Walter, “Genistin (an Isoflavone Glucoside) and its Aglucone, Genistein, from Soybeans”, J. Am. Chem. Soc., 63(12), 3273-3276 (1941)). Of the soy isoflavones, genistein ranks first in the number of experimental and clinical studies performed. Genistein itself is found in low concentrations in soybeans, but at high concentrations in fermented soy-derived foods such as miso and natto. It is also widely distributed in leguminous plant foods as well as in seeds, fruits, and vegetables such as alfalfa and clover sprouts, broccoli, cauliflower, sunflower, barley meal, caraway, and clover seeds. It is widely available in the US as a nutritional supplement, frequently in combination with other isoflavones such as genistin (genistein 7-O-β-D-glucoside) and daidzein.

Many derivatives of genistein have been synthesized: Spagnuolo et al., “Genistein and Cancer: Current Status, Challenges, and Future Directions”, Adv. Nutr., 6, 408-419 (2015) say: “On this basis and because of the known beneficial biological effects of genistein, chemists have been so far encouraged to synthesize many derivatives of this compound, with improved pharmacologic profile. For instance, FA-esterified, 6-carboxymethyl, nitroxy, 7-O-heterocycle, 7-O-β-D-glucoside and 7-O-β-D-glucuronic acid, halogenated, deoxybenzoin, benzylated, hydroxylated, esterified, benzosulfonate, dimethylaminomethyl, phenoxyalkylcarboxylic acid, glycoconjugate, and alkylbenzylamine derivatives of genistein have been reported to date”, citing 15 references. As a few examples of the genistein derivatization literature, Grynkiewicz et al., “X-ray and 13C CP MAS investigations of structure of two genistein derivatives”, J. Mol. Struct., 694, 121-129 (2004) report the synthesis of the 7-O-(t-butyldimethylsilyl) ether and 7-O-(tetrabutylammonium) salt of genistein, useful as intermediates to other 7-substituted genistein derivatives. Rusin et al., “Synthetic conjugates of genistein affecting proliferation and mitosis of cancer cells”, Bioorg. Med. Chem., 19, 295-305 (2011), report the syntheses of 7-O-β-hydroxyalkylgenisteins and conjugates. Szeja et al., “Selective alkylation of genistein and daidzein”, Chem. Biol. Interface, 3, 95-106 (2013), describe regioselective 7-O- and 4-O-alkylations of genistein, including the preparation of dialkylated derivatives; while Byczek et al., “Genistein Derivatives Regioisomerically Substituted at 7-O— and 4′-O— Have Different Effect on the Cell Cycle”, J. Chem., 2013, 191563, describe further regioisomerically substituted genistein derivatives. Castro et al., “Synthesis of Lipophilic Genistein Derivatives and Their Regulation of IL-12 and TNF-α in Activated J774A.1 Cells”, Chem. Biol. Drug Des., 79, 347-352 (2012) report the synthesis of both acylated and alkylated derivatives of genistein, including general procedures for the synthesis of these classes of genistein derivatives. The genistein sulfates are known as human metabolites of genistein (see, e.g., Pugazhendhi et al., “Effect of sulphation on the oestrogen agonist activity of the phytoestrogens genistein and daidzein in MCF-7 human breast cancer cells”, J. Endocrinol., 197, 503-515 (2008)); and the regioselective preparation of genistein 7-O-sulfate and 4′-O-sulfate is described in Shimohira et al., “Regioselective production of sulfated polyphenols using human cytosolic sulfotransferase (SULT)-expressing Escherichia coli cells”, J. Biosci. Bioeng., 124(1), 84-90 (2017). The preparation of genistein 7-O-phosphate and related compounds by biotransformation is described in, e.g., Japanese Patent Application Publication No. 2002-238594 (Mitsukan Group K.K.); Hsu et al., “Soy isoflavone-phosphate conjugates derived by cultivating Bacillus subtilis var. natto BCRC 80517 with isoflavone”, Food Res. Int'l, 53, 487-495 (2013); and US Patent Application Publication No. 2017/0305944 (National Taiwan University); while Wang et al., “Osteoprotective effect of genistein 7-O-phosphate, a derivative of genistein with high bioavailability, in ovariectomized rats”, J. Funct. Foods, 58, 171-179 (2019), describe genistein 7-O-phosphate as being “practically water soluble and more bioavailable than aglyconic genistein”. Crystalline genistein monohydrate and crystalline genistein salts, such as the sodium, potassium, calcium, magnesium, L-lysine, and N-methylglucamine salts, are known in, for example, PCT International Publication No. WO 2010/068861 A1, “Crystalline forms of genistein”, Axcentua Pharmaceuticals AB.

Genistein influences multiple biochemical functions in living cells: it is a full agonist of estrogen receptor β (ERβ; EC₅₀=7.6 nM), and, to about a 20-fold lesser extent, a full or partial agonist of ERα; an agonist of the G protein-coupled estrogen receptor (affinity=133 nM); and activator of peroxisome proliferator-activated receptors; and an inhibitor of several tyrosine kinases, the EFGR kinase and the BCR-ABL protein specific tyrosine kinase. Genistein is a well-studied anticancer agent, reportedly active through multiple mechanisms, including as an angiogenesis inhibitor, a DNA topoisomerase II inhibitor, and as a tyrosine kinase inhibitor. Reports on the effect of genistein on appetite are mixed. PCT International Publication No. WO 2004/069774 (St. Andrews University) discloses isoflavonoid phytoestrogens, such as genistein and daidzein, as implicated in the prevention of cardiovascular disease. WO 00/30665 (Nutri Pharma AS) discloses a composition comprising genistein for use in treating cardiovascular diseases including hypercholesterolemia, hypertriglyceridemia, other hyperlipidemias, arteriosclerosis, arteriolosclerosis, coronary heart disease, angina pectoris, thrombosis, myocardial infarction, and hypertension. US Application Publication No. 2007/0207225 (Squadrito) discloses a composition comprising substantially pure genistein for improving cardiovascular function in mammals, where the composition is said to reduce the risk of at least one of atherosclerosis, coronary heart disease, peripheral vascular disease, myocardial infarction, and carotid stenosis.

Inflammation, while primarily a protective biological response triggered by harmful stimuli, can become detrimental when persistent or systemic. Genistein's anti-inflammatory properties are primarily modulated through:
(a) the NF-κB pathway: genistein effectively inhibits the nuclear factor-kappa B (NF-κB) pathway, a principal inflammatory response regulator. By curtailing the activation and nuclear translocation of NF-κB, genistein suppresses the transcription of pro-inflammatory genes and the subsequent production of pro-inflammatory cytokines;
(b) COX-2 inhibition: genistein also inhibits cyclooxygenase-2 (COX-2), an enzyme integral to the synthesis of pro-inflammatory prostaglandins. This inhibition results in reduced inflammation and pain; and
(c) the MAPK pathway: genistein also impacts the Mitogen-Activated Protein Kinase (MAPK) pathway, another major regulator of inflammatory responses.

Oxidative stress, characterized by an imbalance between free radical production and the body's antioxidant defenses, can lead to cell and tissue damage. Genistein's antioxidant properties are two-fold:
(a) free radical scavenging: genistein acts as a direct antioxidant by neutralizing free radicals, curtailing the cellular damage; and
(b) Nrf2 pathway activation: genistein augments the body's inherent antioxidant defenses by activating the Nrf2 pathways, upregulating the expression of antioxidant genes.

Genistein has undergone various clinical evaluations to assess its safety and efficacy profile. In a Phase III trial, genistein has been also tested in children with Sanfillipo syndrome, median age 7.2 years, at a dose of 160 mg/kg/day (EudraCT Number 2013-001479-18) for 12 months. No treatment-specific severe adverse effects were reported. In another trial, patients with bladder cancer were safely treated with 300 and 600 mg of genistein daily for 21 days, providing valuable insights into its tolerability. In this trial, genistein was well tolerated, with no to mild side effects, and dose was not considered dose-limiting. Gastrointestinal adverse events across all treatment groups included constipation, diarrhea, nausea, and heartburn, whose severity or frequency were also not dose dependent. A more extensive investigation was conducted with healthy volunteers, where genistein, produced as an amorphous solid dispersion by hot melt extrusion to improve bioavailability, was administered in single ascending doses ranging from 500 to 3000 mg (6 participants per dose). This was followed by a multiple single dose regimen of 3000 mg, administered over six consecutive days (10 participants). No significant adverse effects were reported, other than instances of diarrhea, headache or nausea at the highest dosages (2000 and 3000 mg). In long-term studies on postmenopausal women (389 participants), the compound was administered at a dose of 54 mg per day over 24-months. Subsequently, a sub-cohort (138 participants) continued therapy for an additional year. After 36 months, genistein demonstrated no significant changes in mammographic breast density, endometrial thickness, or the expression of BRCA1 and BRCA2, while reducing sister chromatid exchange compared to placebo. Importantly, it led to greater increases in bone mineral density at both the femoral neck and lumbar spine, as well as significant alterations in several bone-related biomarkers. No differences in discomfort or adverse events were reported between participants, further substantiating the safety profile of genistein.

While genistein is widely available in the United States as a dietary supplement, genistein is known to have low solubility and therefore low bioavailability, even though it has high cell wall permeability; and various attempts have been made to increase bioavailability of genistein both by chemical modification (see the second paragraph of this subsection) and by physical means, such as by milling into submicron particles and administration as a suspension (see PCT International Publications No. WO 2012/068140 A1, “Nanoparticle isoflavone compositions & methods of making and using the same”, and WO 2015/081018 A1, “Suspension compositions of physiologically active phenolic compounds & methods of making and using the same”, both to Humanetics Corporation).

The disclosures of the documents referred to in this application are incorporated into this application by reference.

### SUMMARY OF THE INVENTION

This invention is new genistein phosphate derivatives, pharmaceutical compositions containing them, and their uses.

Because administration of genistein has shown activity against many conditions in both in vitro and in vivo assays, and because the genistein phosphate derivatives of this invention show greater solubility and bioavailability than genistein, these genistein phosphate derivatives are expected to be readily bioavailable, readily capable of being formulated into pharmaceutical formulations, and readily capable of treating conditions that are known or suggested as being treatable by genistein or a genistein derivative. In particular, the genistein phosphate derivatives of this invention are expected to be useful in the treatment of cardiac and inflammatory conditions such as dilated cardiomyopathy, Duchenne muscular dystrophy, heart failure, inflammatory bowel disease/ulcerative colitis/Crohn's disease, obesity and obesity-induced diabetes, osteoarthritis, pancreatitis, pancreatic ductal adenocarcinoma, rheumatoid arthritis, systemic vasculitis, and transthyretin amyloid cardiomyopathy. They are also expected to be useful in the treatment of Down syndrome.

Certain embodiments of this invention are characterized by the specification and by the features of Claims 1 to 20 of this application as filed.

### DETAILED DESCRIPTION OF THE INVENTION

### Definitions

“Genistein” is described in the section entitled “Genistein” in the DESCRIPTION OF THE

### RELATED ART

A “genistein phosphate derivative” means a compound of formula I or II:

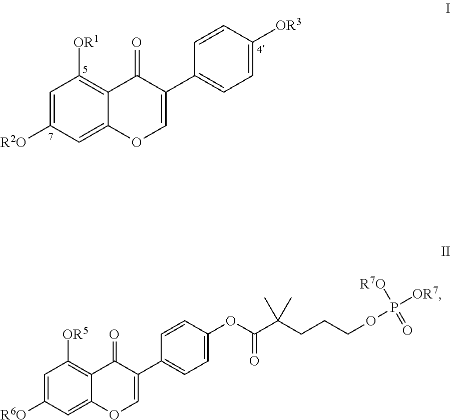

or a salt, especially a pharmaceutically acceptable salt, thereof, where:
in formula I:
each of R¹, R², and R³ independently is selected from the group consisting of H, C₁₋₆ alkyl, and (R⁴O)₂P(O)CH₂—, provided that at least one of R¹, R², and R³ is (R⁴O)₂P(O)CH₂—; and
each R⁴ independently is selected from the group consisting of H and C₁₋₆ alkyl; and
in formula II:
each of R⁵ and R⁶ independently is selected from the group consisting of H, C₁₋₆ alkyl, and (R⁷O)₂P(O)CH₂—, and each R⁷ independently is selected from the group consisting of H and C₁₋₆ alkyl.

A “condition treatable by genistein or a genistein derivative” is any condition in which genistein or a genistein derivative, such as the derivatives mentioned in the section entitled “Genistein” in the DESCRIPTION OF THE RELATED ART, have been tested or suggested to provide treatment (as “treatment” is defined below) for that condition. Such conditions include the conditions mentioned in the section entitled “Genistein” in the DESCRIPTION OF THE RELATED ART, and in the Examples, such as cardiovascular conditions, inflammatory conditions, and conditions requiring osteoprotection.

A “therapeutically effective amount” of a genistein phosphate derivative means that amount of the genistein phosphate derivative which, when the genistein phosphate derivative is administered to a human for a condition treatable by that genistein phosphate derivative (such as the conditions mentioned in the SUMMARY OF THE INVENTION and the Examples), or by genistein or a genistein derivative, is sufficient to effect treatment for the condition.

“Treating” or “treatment” of such a condition in a human includes one or more of:
(1) preventing or reducing the risk of developing the condition, i.e., causing the clinical symptoms of the condition not to develop in a subject who may be predisposed to the condition but who does not yet experience or display symptoms of the condition (i.e., prophylaxis);
(2) inhibiting the condition, i.e., arresting or reducing the development of the condition or its clinical symptoms; and
(3) relieving the condition, i.e., causing regression, reversal, or amelioration of the condition or reducing the number, frequency, duration or severity of its clinical symptoms.
   “Treatment” does not necessarily imply “cure” or complete treatment, e.g., treatment of all clinical symptoms of the condition, though “treatment” may include “cure”. Rather, “treatment” implies the provision of clinical benefit by the administration of the genistein phosphate derivative when compared to non-administration of the genistein phosphate derivative; and treatment may also be assessed by improvement in biological markers of the condition being treated. The therapeutically effective amount for a particular subject varies depending upon the health and physical condition of the subject to be treated, the condition and its extent, the assessment of the medical situation, and other relevant factors. It is expected that the therapeutically effective amount will fall in a relatively broad range that can be determined through routine trial.

“Comprising” or “containing” and their grammatical variants are words of inclusion and not of limitation and mean to specify the presence of stated components, groups, steps, and the like but not to exclude the presence or addition of other components, groups, steps, and the like. Thus “comprising” does not mean “consisting of”, “consisting substantially of”, or “consisting only of”;
and, for example, a formulation “comprising” a compound must contain that compound but also may contain other active ingredients and/or excipients.
   The Genistein Phosphate Derivatives and their Synthesis

The genistein phosphate derivatives of this invention are the compounds of formula I and II:

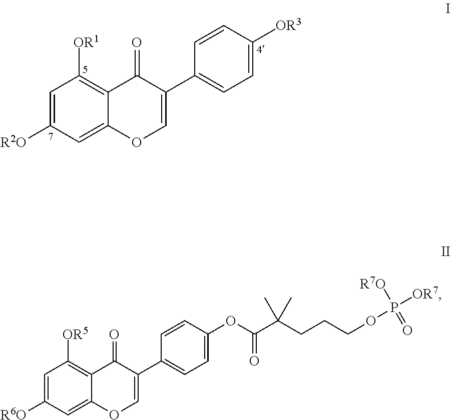

and the salts, especially the pharmaceutically acceptable salts, thereof, where:
in formula I:
each of R¹, R², and R³ independently is selected from the group consisting of H, C₁₋₆ alkyl, and (R⁴O)₂P(O)CH₂—, provided that at least one of R¹, R², and R³ is (R⁴O)₂P(O)CH₂—; and
each R⁴ independently is selected from the group consisting of H and C₁₋₆ alkyl; and
in formula II:
each of R⁵ and R⁶ independently is selected from the group consisting of H, C₁₋₆ alkyl, and (R⁷O)₂P(O)CH₂—, and each R⁷ independently is selected from the group consisting of H and C₁₋₆ alkyl.

In one embodiment of these compounds, the C₁₋₆ alkyl is selected from the group consisting of methyl, ethyl, propyl, isopropyl, and butyl.

Compounds of interest within these genistein phosphate derivatives include:
(a) the compounds of formula I,
(b) within (a), the compounds of formula I where R¹ is H,
(c) within (a) or (b), the compounds of formula I where each R⁴ is H, and
(d) within (c), the compounds that are (4-(5,7-dihydroxy-4-oxo-4H-chromen-3-yl)phenoxy)-methyl dihydrogen phosphate, ((5-hydroxy-3-(4-hydroxyphenyl)-4-oxo-4H-chromen-7-yl)oxy)-methyl dihydrogen phosphate, or ((5-hydroxy-4-oxo-3-(4-((phosphonooxy)methoxy)phenyl)-4H-chromen-7-yl)oxy)methyl dihydrogen phosphate, especially ((5-hydroxy-3-(4-hydroxyphenyl)-4-oxo-4H-chromen-7-yl)oxy)methyl dihydrogen phosphate;
and their salts, such as their pharmaceutically acceptable salts, especially the compounds in non-salt form; or
(e) the compounds of formula II,
(f) within (e), the compounds of formula II where R⁵ and R⁶ are both H,
(g) within (e) or (f), the compounds of formula II where each R⁷ is H, and
(h) within (g), the compound that is 4-(5,7-dihydroxy-4-oxo-4H-chromen-3-yl)phenyl 2,2-dimethyl-5-(phosphonooxy)pentanoate;
and their salts, such as their pharmaceutically acceptable salts, especially the compounds in non-salt form.

Salts, especially pharmaceutically acceptable salts, of the genistein phosphate derivatives are included in this application, as mentioned in the definition of “genistein phosphate derivatives” above, and are useful in the methods described in this application. These salts are preferably formed with pharmaceutically acceptable acids or bases, as appropriate. See, for example, “Handbook of Pharmaceutically Acceptable Salts”, Stahl and Wermuth, eds., Verlag Helvetica Chimica Acta, Zurich, Switzerland, for an extensive discussion of pharmaceutical salts, their selection, preparation, and use. Unless the context requires otherwise, any reference to genistein is a reference both to the compound and to its salts. Genistein can exist as the non-salt compound, but salts with alkali metals and nitrogenous bases (e.g., tromethamine) are known, for example, PCT Publication No. WO 2010/068861, referred to previously, discloses both alkali metal and nitrogenous base salts.

The genistein phosphate derivatives may be prepared by conventional methods of organic synthesis well-known to those of ordinary skill in the art, having regard to that knowledge and the known methods of synthesis of genistein derivatives as exemplified in the documents referred to in the section entitled “Genistein” in the DESCRIPTION OF THE RELATED ART, and others, and as exemplified in the Examples below. Typically, for compounds of formula I, genistein is reacted with a dialkyl ester of (chloromethyl)phosphonic acid, such as di-tert-butyl (chloromethyl)phosphate, in an aprotic polar solvent such as N,N-dimethylformamide, in the presence of an excess of an excess of a base such as potassium tert-butoxide or cesium carbonate, and a halide such as sodium iodide or tert-butylammonium bromide to form an esterified phosphonooxymethyl ether of genistein, followed by hydrolysis to remove the phosphonate alkyl groups. Since the relative reactivity of the three hydroxy groups of genistein to ether formation is lowest at the 5-position, preparation of the compounds of formula I where R¹ is (HO)₂P(O)CH₂— may require protection of one or both of the 7- and 4′-positions with hydroxy protecting groups such as methoxymethyl (as described in Example 1 below) or benzyl if multiple substitution is not desired. Purification of the intermediate and final products may be accomplished by the usual methods, such as chromatography, slurrying, and recrystallization.

In each case, confirmation of the identity of the product may be obtained using conventional methods of analysis, such as nuclear magnetic resonance and mass spectrometry.

Subjects Suitable for Treatment by this Invention

Suitable subjects for treatment by the compounds, pharmaceutical compositions, and methods of treatment, etc. of the present application are humans suffering from conditions treatable by the genistein phosphate derivatives of this invention (as mentioned in the SUMMARY OF THE INVENTION and described in the Examples), or by genistein or a genistein derivative, or considered likely to suffer from such conditions.

### Formulation and Administration

The genistein phosphate derivatives may be administered by any route suitable to the subject being treated and the nature of the subject's condition. Routes of administration include oral administration (generally preferred, if available); administration by injection, including intravenous, intraperitoneal, intramuscular, and subcutaneous injection; by transmucosal (e.g., intranasal, buccal, sublingual, rectal, or vaginal) or transdermal (topical) delivery; and the like. Formulations may be oral formulations (e.g., tablets, capsules, or oral solutions or suspensions); injectable formulations (e.g., solutions); and formulations designed to administer the drug across mucosal membranes or transdermally. Suitable formulations for each of these methods of administration may be found, for example, in “Remington: The Science and Practice of Pharmacy”, 20th ed., Gennaro, ed., Lippincott Williams & Wilkins, Philadelphia, Pa., U.S.A. Because the genistein phosphate derivatives are generally orally available, typical formulations will be oral, and typical dosage forms will be tablets or capsules for oral administration. Intravenous formulations may be particularly applicable for administration to acutely ill subjects, such as those subjects who may be hospitalized for treatment.

Depending on the intended mode of administration, the pharmaceutical compositions may be in the form of solid, semi-solid or liquid dosage forms, preferably in unit dosage form suitable for single administration of a precise dosage. In addition to a therapeutically effective amount of the genistein phosphate derivative, the compositions may contain suitable pharmaceutically-acceptable excipients, including adjuvants which facilitate processing of the active compounds into preparations which can be used pharmaceutically. “Pharmaceutically acceptable excipient” refers to an excipient or mixture of excipients which does not interfere with the effectiveness of the biological activity of the active compound(s) and which is not toxic or otherwise undesirable to the subject to which it is administered.

For solid compositions, conventional pharmaceutically acceptable excipients include, for example, pharmaceutical grades of mannitol, lactose, starch, magnesium stearate, sodium saccharin, talc, cellulose, glucose, sucrose, magnesium carbonate, and the like. Liquid pharmacologically administrable compositions can, for example, be prepared by dissolving, dispersing, etc., an active compound as described herein and optional pharmaceutical adjuvants in water or an aqueous excipient, such as, for example, water, saline, aqueous dextrose, and the like, to form a solution or suspension. If desired, the pharmaceutical composition to be administered may also contain minor amounts of nontoxic auxiliary excipients such as wetting or emulsifying agents, pH buffering agents and the like, for example, sodium acetate, sorbitan monolaurate, triethanolamine sodium acetate, triethanolamine oleate, etc. When liquid suspensions are used, the active agent may be combined with emulsifying and suspending excipients. If desired, flavoring, coloring and/or sweetening agents may be added as well. Other optional excipients for incorporation into an oral formulation include preservatives, suspending agents, thickening agents, and the like.

Typically, a pharmaceutical composition of one of the genistein phosphate derivatives is packaged in a container with a label, or instructions, or both, indicating use of the pharmaceutical composition in the treatment of a condition treatable by a genistein phosphate derivative of this invention.

A suitable amount of a genistein phosphate derivatives for oral dosing (subjects with conditions treatable by the genistein phosphate derivatives may well be taking other therapies in addition to the genistein phosphate derivatives of this application) may be within a range selected from about 0.1-100, 0.2-80, 0.3-60, 0.4-40, or 0.5-20 mg/Kg/day. Alternatively, a suitable amount of the genistein phosphate derivative may be in a range selected from about 1-2,500, 2-2,000, 5-1,500, 10-1,000, 15-750, or 20-500 mg twice/day.

A person of ordinary skill in the art of the treatment of conditions treatable by genistein or a genistein derivative will be able to ascertain a therapeutically effective amount of the genistein phosphate derivative for a particular subject and extent of the condition, to achieve a therapeutically effective amount without undue experimentation and in reliance upon personal knowledge and the disclosure of this application.

### SYNTHESIS EXAMPLES

Glossary: ACN: acetonitrile; DCM: dichloromethane; DMF: N,N-dimethylformamide; DMSO: dimethyl sulfoxide; EtOAc: ethyl acetate; LCMS: liquid chromatography-mass spectrometry; NMR: nuclear magnetic resonance; TBAB: tert-butylammonium bromide; TFA: trifluoroacetic acid; THF: tetrahydrofuran; TLC: thin layer chromatography.

### Example 1: (4-(5,7-Dihydroxy-4-oxo-4H-chromen-3-yl)phenoxy)methyl dihydrogen phosphate, genistein 4′-((phosphonooxy)methyl) ether, Compound 1

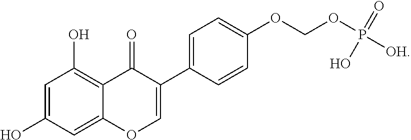 [SMILES: O=c1c(-c2ccc(OCOP(=O)(O)O)cc2)coc2cc(O)cc(O)c12]

Step 1: 5-Hydroxy-3-(4-hydroxyphenyl)-7-(methoxymethoxy)-4H-chromen-4-one. To a solution of genistein (10.0 g, 37.0 mmol, 1.0 eq) in DMF (100 mL) was added potassium iodide (1.23 g, 7.40 mmol, 0.2 eq) and potassium hydroxide (4.15 g, 74.0 mmol, 2.0 eq), then chloro(methoxy)methane (5.11 g, 63.5 mmol, 4.82 mL, 1.7 eq) was added at 0° C. The mixture was stirred at 20° C. for 12 hours. LCMS showed that the starting material had been consumed completely and that one main peak with the desired mass was detected. The reaction mixture was poured into water (100 mL) at 20° C., and the pH adjusted to around 3 by addition of 1M hydrochloric acid. The reaction mixture was extracted with DCM (3×200 mL). The combined organic extracts were washed with brine (200 mL), dried over sodium sulfate, filtered and concentrated under vacuum to give a residue which was purified by flash silica gel chromatography using a gradient of ethyl acetate/petroleum ether from 0:1 to 1:1. 5-Hydroxy-3-(4-hydroxyphenyl)-7-(methoxymethoxy)-4H-chromen-4-one (8.00 g, 25.5 mmol, 69% yield) was obtained as a yellow solid. (M+H)⁺=314.9 (LCMS).

Step 2: Di-tert-butyl ((4-(5-hydroxy-7-(methoxymethoxy)-4-oxo-4H-chromen-3-yl)phenoxy)methyl) phosphate. To a solution of 5-hydroxy-3-(4-hydroxyphenyl)-7-(methoxymethoxy)-4H-chromen-4-one (2.00 g, 6.36 mmol, 1.0 eq) in DMF (30 mL) was added sodium hydride (382 mg, 9.55 mmol, 60% purity, 1.5 eq) and TBAB (123 mg, 382 mol, 0.06 eq); then di-tert-butyl (chloromethyl)phosphate (1.65 g, 6.36 mmol, 1.0 eq) was added at 0° C. and the mixture was stirred at 50° C. for 4 hours. TLC (1:1 ethyl acetate/petroleum ether, Rf=0.3) showed that the starting material was consumed completely and one new spot formed. The reaction mixture was poured into saturated aqueous ammonium chloride solution (20 mL) at 20° C., and then extracted with ethyl acetate (2×20 mL). The combined organic extracts were washed with brine (20 mL), dried over sodium sulfate, filtered and concentrated under vacuum to give a residue which was purified by flash silica gel chromatography using a gradient of ethyl acetate/petroleum ether from 0:1 to 1:1. Di-tert-butyl ((4-(5-hydroxy-7-(methoxymethoxy)-4-oxo-4H-chromen-3-yl)-phenoxy)methyl) phosphate (1.50 g, 2.72 mmol, 43% yield) was obtained as a yellow oil. (M+Na)⁺=559.2 (LCMS).

Step 3: (4-(5,7-Dihydroxy-4-oxo-4H-chromen-3-yl)phenoxy)methyl dihydrogen phosphate. To a solution of di-tert-butyl ((4-(5-hydroxy-7-(methoxymethoxy)-4-oxo-4H-chromen-3-yl)-phenoxy)methyl) phosphate (1.10 g, 2.05 mmol, 1.0 eq) in DCM (22 mL) was added TFA (3.28 g, 28.7 mmol, 2.13 mL, 14 eq) and the mixture was stirred at 0° C. for 0.5 hour. LCMS showed that the starting material had been consumed completely and the desired mass was detected. The mixture was filtered and purified by preparative HPLC (Waters Xbridge Prep OBD C18 column (150×40 mm, 10 μm); flow rate: 25 mL/min; gradient: 1%-20% B over 6 minutes; mobile phase A: 10 mM aqueous ammonium carbonate, mobile phase B: ACN). (4-(5,7-Dihydroxy-4-oxo-4H-chromen-3-yl)phenoxy)methyl dihydrogen phosphate (119 mg, 302 mol, 15% yield) was obtained as a white solid. (M+H)⁺=381.1 (LCMS); ¹H NMR (400 MHz, DMSO): δ 8.24 (s, 1H), 7.41-7.39 (d, J=8.4 Hz, 2H), 7.11-7.09 (d, J=8.8 Hz, 2H), 6.41 (s, 1H), 6.15 (s, 1H), 5.41-5.39 (d, J=8.0 Hz, 2H).

### Example 2: ((5-Hydroxy-3-(4-hydroxyphenyl)-4-oxo-4H-chromen-7-yl)oxy)methyl dihydrogen phosphate, genistein 7-((phosphonooxy)methyl) ether, Compound 2

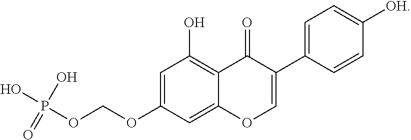 [SMILES: O=c1c(-c2ccc(O)cc2)coc2cc(OCOP(=O)(O)O)cc(O)c12]

Step 1: Di-tert-butyl (((5-hydroxy-3-(4-hydroxyphenyl)-4-oxo-4H-chromen-7-yl)oxy)-methyl) phosphate. To a solution of genistein (4.00 g, 14.8 mmol, 1.0 eq) in DMF (40 mL) was added potassium tert-butoxide (1 M in THF, 29.6 mL, 2.0 eq) and sodium iodide (2.22 g, 14.8 mmol, 1.0 eq); then di-tert-butyl(chloromethyl)phosphate (9.57 g, 37.0 mmol, 2.5 eq) was added and the mixture was stirred at 20° C. for 12 hours. LCMS showed that the starting material had been consumed completely and the desired mass was detected. The mixture was poured into water (300 mL), then extracted with ethyl acetate (3×300 mL). The combined organic extracts were washed with brine (300 mL), dried over sodium sulfate, filtered and concentrated under vacuum to give a residue which was purified by flash silica gel chromatography using a gradient of ethyl acetate/petroleum ether from 0:1 to 1:1. Di-tert-butyl (((5-hydroxy-3-(4-hydroxyphenyl)-4-oxo-4H-chromen-7-yl)oxy)methyl) phosphate (1.85 g, 3.76 mmol, 25% yield) was obtained as a yellow solid. (M+Na)⁺=515.2 (LCMS).

Step 2: ((5-Hydroxy-3-(4-hydroxyphenyl)-4-oxo-4H-chromen-7-yl)oxy)methyl dihydrogen phosphate. To a solution of di-tert-butyl (((5-hydroxy-3-(4-hydroxyphenyl)-4-oxo-4H-chromen-7-yl)oxy)methyl) phosphate (0.50 g, 1.02 mmol, 1.0 eq) in DCM (5.0 mL) was added TFA (0.5 mL), and the mixture was stirred at 20° C. for 1 hour. LCMS showed that the starting material had been consumed completely and the desired mass was detected. The mixture was filtered and the filter cake was purified by preparative HPLC (Phenomenex Luna C18 column (75×30 mm, 3 μm); flow rate: 25 mL/min; gradient: 10%-40% B over 8 minutes; mobile phase A: 0.1% aqueous TFA, mobile phase B: ACN). ((5-Hydroxy-3-(4-hydroxyphenyl)-4-oxo-4H-chromen-7-yl)oxy)methyl dihydrogen phosphate (163 mg, 429 mol, 42% yield) was obtained as a yellow solid. (M+H)⁺=381.1 (LCMS); ¹H NMR (400 MHz, DMSO): δ 12.95 (s, 1H), 9.61 (s, 1H), 8.44 (s, 1H), 7.41-7.38 (d, J=8.4 Hz, 2H), 6.83-6.81 (m, 3H), 6.53 (s, 1H), 5.67-5.64 (d, J=12 Hz, 2H).

### Example 3: ((5-Hydroxy-4-oxo-3-(4-((phosphonooxy)methoxy)phenyl)-4H-chromen-7-yl)oxy)methyl dihydrogen phosphate, genistein 4′,7-bis((phosphonooxy)methyl) ether, Compound 3

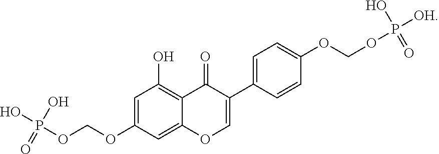 [SMILES: O=c1c(-c2ccc(OCOP(=O)(O)O)cc2)coc2cc(OCOP(=O)(O)O)cc(O)c12]

Step 1: Di-tert-butyl (((5-hydroxy-3-(4-hydroxyphenyl)-4-oxo-4H-chromen-7-yl)oxy)-methyl) phosphate. To a solution of genistein (4.40 g, 16.3 mmol, 1.0 eq) in DMF (50 mL) was added potassium tert-butoxide (1 M in THF, 32.6 mL, 2.0 eq) and sodium iodide (2.44 g, 16.3 mmol, 1.0 eq); then di-tert-butyl(chloromethyl)phosphate (10.5 g, 40.7 mmol, 2.5 eq) was added at 0° C. and the mixture was stirred at 20° C. for 4 hours. TLC (ethyl acetate/petroleum ether=1:1, Rf=0.4) showed that the starting material had been consumed completely. The mixture was poured into water (300 mL), then extracted with ethyl acetate (3×300 mL), washed with brine (300 mL), dried over sodium sulfate, filtered and concentrated under vacuum to give a residue, which was purified by flash silica gel chromatography using a gradient of ethyl acetate/petroleum ether from 0:1 to 4:5. Di-tert-butyl (((5-hydroxy-3-(4-hydroxyphenyl)-4-oxo-4H-chromen-7-yl)-oxy)methyl) phosphate (3.20 g, 2.94 mmol, 18% yield) was obtained as a yellow solid. (M+Na)⁺=515.1 (LCMS).

Step 2: Di-tert-butyl (((3-(4-(((di-tert-butoxyphosphoryl)oxy)methoxy)phenyl)-5-hydroxy-4-oxo-4H-chromen-7-yl)oxy)methyl) phosphate. To a solution of di-tert-butyl (((5-hydroxy-3-(4-hydroxyphenyl)-4-oxo-4H-chromen-7-yl)oxy)methyl) phosphate (1.60 g, 3.25 mmol, 1.0 eq) in DMF (50 mL) was added sodium hydride (325 mg, 8.12 mmol, 60% purity, 2.5 eq) and TBAB (62.8 mg, 195 mol, 0.06 eq) and the mixture was stirred at 0° C. for 0.5 hours, then di-tert-butyl (chloromethyl) phosphate (1.68 g, 6.50 mmol, 2.0 eq) was added and the mixture was stirred at 50° C. for 5 hours. TLC (ethyl acetate/petroleum ether=1:1, Rf=0.4) showed that the starting material had been consumed completely. The reaction mixture was poured into saturated aqueous ammonium chloride (30 mL) at 20° C., and then extracted with ethyl acetate (2×15 mL). The combined organic extracts were washed with brine (30 mL), dried over sodium sulfate, filtered and concentrated under vacuum to give a residue, which was purified by flash silica gel chromatography using a gradient of ethyl acetate/petroleum ether from 0:1 to 1:0. Di-tert-butyl (((3-(4-(((di-tert-butoxyphosphoryl)oxy)methoxy)phenyl)-5-hydroxy-4-oxo-4H-chromen-7-yl)oxy)methyl) phosphate (0.80 g, 1.12 mmol, 29% yield) was obtained as a yellow oil. (M+Na)⁺=737.3 (LCMS).

Step 3: ((5-Hydroxy-4-oxo-3-(4-((phosphonooxy)methoxy)phenyl)-4H-chromen-7-yl)-oxy)methyl dihydrogen phosphate. To a solution of di-tert-butyl (((3-(4-(((di-tert-butoxyphosphoryl)oxy)methoxy)phenyl)-5-hydroxy-4-oxo-4H-chromen-7-yl)oxy)-methyl) phosphate (800 mg, 1.12 mmol, 1.0 eq) in DCM (32 mL) was added TFA (1.6 mL) at 0° C., and the mixture was stirred at 0° C. for 0.5 hours. LCMS showed that the starting material had been consumed completely and one main peak with the desired mass was detected. The mixture was filtered and the filter cake was concentrated under vacuum to give a residue which was purified by preparative HPLC (Waters Xbridge Prep OBD C18 column (150×40 mm, 10 μm); flow rate: 25 mL/min; gradient: 1%-5% B over 6 minutes; mobile phase A: 0.05% aqueous ammonia+10 mM aqueous ammonium bicarbonate, mobile phase B: ACN). ((5-Hydroxy-4-oxo-3-(4-((phosphonooxy)methoxy)phenyl)-4H-chromen-7-yl)oxy)methyl dihydrogen phosphate (131 mg, 22% yield) was obtained as a yellow solid. (M+H)⁺=491.1 (LCMS); ¹H NMR (400 MHz, DMSO): δ 8.21 (s, 1H), 7.36-7.34 (d, J=8.4 Hz, 2H), 7.01-6.99 (d, J=8.4 Hz, 2H), 6.64 (s, 1H), 6.38 (s, 1H), 5.38-5.33 (m, 4H).

### Example 4: 4-(5,7-Dihydroxy-4-oxo-4H-chromen-3-yl)phenyl 2,2-dimethyl-5-(phosphonooxy)pentanoate, genistein 4′-(2,2-dimethyl-5-(phosphonooxy)pentanoate), Compound 4

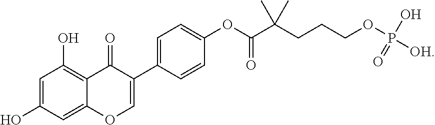 [SMILES: CC(C)(CCCOP(=O)(O)O)C(=O)Oc1ccc(-c2coc3cc(O)cc(O)c3c2=O)cc1]

Step 1: (3-Bromopropoxy)(tert-butyl)diphenylsilane. To a solution of 3-bromopropan-1-ol (11.0 g, 79.1 mmol, 7.16 mL, 1.0 eq) in DCM (100 mL) was added imidazole (8.08 g, 119 mmol, 1.5 eq). (tert-Butyl)chlorodiphenylsilane (23.9 g, 87.1 mmol, 22.3 mL, 1.1 eq) was added at 0° C., and the mixture was stirred at 20° C. for 5 hours. TLC (ethyl acetate/petroleum ether=5:1, Rf=0.8) showed that the starting material had been consumed completely. The reaction mixture was poured into water (100 mL) at 20° C., and then extracted with DCM (2×100 mL). The combined organic extracts were washed with brine (100 mL), dried over sodium sulfate, filtered and concentrated under vacuum to give a residue, which was purified by flash silica gel chromatography using a gradient of ethyl acetate/petroleum ether from 1:100 to 1:10. (3-Bromopropoxy)(tert-butyl)diphenyl-silane (29.0 g, 76.8 mmol, 97% yield) was obtained as a colorless oil.

Step 2: Methyl 5-(((tert-butyl)diphenylsilyl)oxy)-2,2-dimethylpentanoate. To a solution of methyl isobutyrate (8.44 g, 82.7 mmol, 9.48 mL, 1.3 eq) in THF (300 mL) was added lithium N,N′-dimethylamide (2 M, 63.6 mL, 2.0 eq) at −78° C., and the mixture was stirred at −78° C. for 1 hour, then (3-bromopropoxy)(tert-butyl)diphenylsilane (24.0 g, 63.6 mmol, 1.0 eq) was added and the mixture was stirred at 20° C. for 2 hours. TLC (ethyl acetate/petroleum ether, Rf=0.6) showed that the starting material had been consumed completely. The mixture was poured into saturated aqueous ammonium chloride (500 mL) and extracted with EtOAc (2×500 mL). The combined organic extracts were washed with brine (500 mL), dried over sodium sulfate, filtered and concentrated under vacuum to give methyl 5-(((tert-butyl)diphenylsilyl)oxy)-2,2-dimethylpentanoate (19.0 g, 47.7 mmol, 75% yield) as a yellow oil.

Step 3: 5-(((tert-Butyl)diphenylsilyl)oxy)-2,2-dimethylpentanoic acid. To a solution of methyl 5-(((tert-butyl)diphenylsilyl)oxy)-2,2-dimethylpentanoate (9.50 g, 23.8 mmol, 1.0 eq) in THF (20 mL), methanol (20 mL) and water (10 mL) was added lithium hydroxide monohydrate (2.00 g, 47.7 mmol, 2.0 eq) and the mixture was stirred at 20° C. for 24 hours. LCMS showed that 16.9% of the starting material remained and 26.5% of the desired compound was detected. The mixture was filtered and the filtrate was purified by reversed-phase HPLC. 5-(((tert-Butyl)diphenylsilyl)oxy)-2,2-dimethylpentanoic acid (4.00 g, 10.4 mmol, 22% yield) was obtained as a yellow solid. (M+H)⁺=385.2 (LCMS).

Step 4: 4-(5-Hydroxy-7-(methoxymethoxy)-4-oxo-4H-chromen-3-yl)phenyl 5-((tert-butyldiphenylsilyl)oxy)-2,2-dimethylpentanoate. To a solution of 5-((tert-butyldiphenylsilyl)oxy)-2,2-dimethylpentanoic acid (2.80 g, 7.28 mmol, 1.0 eq) and 5-hydroxy-3-(4-hydroxyphenyl)-7-(methoxymethoxy)-4H-chromen-4-one (2.29 g, 7.28 mmol, 1.0 eq) in DMF (30 mL) was added 1-[bis(dimethylamino)methylene]-1H-1,2,3-triazolo[4,5-b]pyridinium-3-oxide hexafluorophosphate (3.32 g, 8.74 mmol, 1.2 eq) and N,N-di(isopropyl)ethylamine (2.35 g, 18.2 mmol, 3.17 mL, 2.5 eq). The mixture was stirred at 50° C. for 12 hours. LCMS showed that 12% of the starting material remained and 19% of the desired compound was detected. The reaction mixture was poured into water (30 mL) at 20° C., and then extracted with EtOAc (2×30 mL). The combined organic extracts were washed with brine (30 mL), dried over sodium sulfate, filtered and concentrated under vacuum to give a residue, which was purified by flash silica gel chromatography using a gradient of ethyl acetate/petroleum ether from 1:100 to 1:20. 4-(5-Hydroxy-7-(methoxymethoxy)-4-oxo-4H-chromen-3-yl)phenyl 5-((tert-butyldiphenylsilyl)oxy)-2,2-dimethylpentanoate (3.00 g, 2.00 mmol, 27% yield) was obtained as a yellow oil. (M+H)⁺=681.4 (LCMS).

Step 5: 4-(5-Hydroxy-7-(methoxymethoxy)-4-oxo-4H-chromen-3-yl)phenyl 5-hydroxy-2,2-dimethylpentanoate. To a solution of 4-(5-hydroxy-7-(methoxymethoxy)-4-oxo-4H-chromen-3-yl)phenyl 5-((tert-butyldiphenylsilyl)oxy)-2,2-dimethylpentanoate (3.00 g, 4.41 mmol, 1.0 eq) in DMF (10 mL) was added triethylamine hydrogen fluoride (6.41 g, 52.9 mmol, 12 eq) at 0° C., and the mixture was stirred at 20° C. for 1 hour. LCMS showed that the starting material had been consumed completely and one main peak with the desired mass was detected. The mixture was filtered and the filtrate purified by preparative HPLC (Waters Xbridge BEH C18 column (250×70 mm, 10 μm); flow rate: 25 mL/min; gradient: 40%-75% B over 18 minutes; mobile phase A: 10 mM aqueous ammonium bicarbonate, mobile phase B: ACN). 4-(5-Hydroxy-7-(methoxymethoxy)-4-oxo-4H-chromen-3-yl)phenyl 5-hydroxy-2,2-dimethylpentanoate (0.43 g, 903 mol, 21% yield) was obtained as a white solid. (M+H)⁺=443.2 (LCMS).

Step 6: 4-(5-Hydroxy-7-(methoxymethoxy)-4-oxo-4H-chromen-3-yl)phenyl 5-((di-tert-butoxyphosphoryl)oxy)-2,2-dimethylpentanoate. To a solution of 4-(5-hydroxy-7-(methoxymethoxy)-4-oxo-4H-chromen-3-yl)phenyl 5-hydroxy-2,2-dimethylpentanoate (0.43 g, 972 mol, 1.0 eq) in DCM (10 mL) was added di-tert-butyl N,N-di(isopropyl)phosphoramidite (863 mg, 3.11 mmol, 3.2 eq); then 2H-tetrazole (252 mg, 3.60 mmol, 319 μL, 3.7 eq) was added and the mixture was stirred at 20° C. for 0.5 hour. Aqueous hydrogen peroxide (0.61 g, 5.38 mmol, 517 μL, 30% purity, 5.5 eq) was added at 0° C. and the mixture was stirred at 20° C. for 0.5 hour. LCMS showed that the starting material had been consumed completely and one main peak with the desired mass was detected. The mixture was poured into saturated aqueous sodium sulfite (20 mL), then extracted with EtOAc (2×10 mL). The combined organic extracts were washed with brine (10 mL), dried over sodium sulfate and concentrated under vacuum to give a residue, which was purified by preparative HPLC (Waters Xbridge Prep OBD column (150×40 mm, 10 μm); flow rate: 25 mL/min; gradient: 65%-95% B over 8 minutes; mobile phase A: 0.05% aqueous ammonia+10 mM aqueous ammonium bicarbonate, mobile phase B: ACN). 4-(5-Hydroxy-7-(methoxymethoxy)-4-oxo-4H-chromen-3-yl)phenyl 5-((di-tert-butoxyphosphoryl)oxy)-2,2-dimethylpentanoate (0.30 g, 468 mol, 48% yield) was obtained as a brown solid. (M+Na)⁺=657.4 (LCMS).

Step 7: 4-(5,7-Dihydroxy-4-oxo-4H-chromen-3-yl)phenyl 2,2-dimethyl-5-(phosphonooxy)pentanoate. To a solution of 4-(5-Hydroxy-7-(methoxymethoxy)-4-oxo-4H-chromen-3-yl)phenyl 5-((di-tert-butoxyphosphoryl)oxy)-2,2-dimethylpentanoate (0.3 g, 468 mol, 1.0 eq) in DCM (12 mL) was added TFA (1.2 mL) at −78° C. The mixture was stirred at 20° C. for 1 hour. LCMS showed that the starting material had been consumed completely and one main peak with the desired mass was detected. The mixture was filtered and the filtrate was purified by preparative HPLC (Waters Xbridge Prep OBD C18 column (150×40 mm, 10 μm); flow rate: 25 mL/min; gradient: 10%-40% B over 6 minutes; mobile phase A: 0.05% aqueous ammonia+10 mM aqueous ammonium bicarbonate, mobile phase B: ACN). 4-(5,7-Dihydroxy-4-oxo-4H-chromen-3-yl)phenyl 2,2-dimethyl-5-(phosphonooxy)pentanoate (39.4 mg, 82.3 mol, 17% yield) was obtained as a white solid. (M+H)⁺=479.2 (LCMS); ¹H NMR (400 MHz, DMSO): δ 8.25 (s, 1H), 7.49-7.47 (d, J=8.4 Hz, 2H), 7.10-7.05 (m, 2H), 6.49 (s, 1H), 6.27 (s, 1H), 3.74-3.61 (m, 2H), 1.70-1.66 (m, 2H), 1.58-1.54 (m, 2H), 1.26 (s, 6H).

### Example 5: Polymorph and Salt Screening of Compound 2

Solubility testing of compound 2 was carried out by adding 20 μL aliquots (to a maximum of 1 mL) of selected solvents (water, methanol, DCM, and THF) to about 5 mg samples of compound 2 at 25 C with vortexing and sonication; and the approximate solubility determined by visual observation. Similar testing was carried out at 50 C, using about 10 mg samples of compound 2. Compound 2 had a solubility in water of less than 5 mg/mL at 25° C. and approximately 5 mg/mL at 50° C.; in methanol of more than 250 mg/mL at both 25 and 50° C.; in DCM of less than 5 mg/mL at 25° C.; and in THF of 125-250 mg/mL at both 25 and 50° C.

A sample of compound 2, purified by slurrying in 1:1 THF/acetone and isolation of the solids by centrifugation and drying, gave crystalline material of 99.0% purity by HPLC, with no residual solvents detected by proton NMR. This material, Pattern A, had a decomposition point of approximately 170° C. by differential scanning calorimetry, a weight loss of approximately 1.6% by thermogravimetric analysis below the decomposition point, and an X-ray powder diffraction pattern (CuKα radiation) with the six largest peaks (intensities in parentheses) at 11.80 (40), 17.2° (54), 18.9° (56), 20.3° (65), 21.4° (100), and 22.7° (44) 20, with angles±0.2° 20. The material is a hygroscopic anhydrate. Crystallizations of samples of this material by evaporation of solvents (methanol, water, THF, and 7:3 methanol/water) under nitrogen flow gave the same crystalline form. A second crystalline form, Pattern B, with a decomposition point of approximately 181° C., and having the largest X-ray powder diffraction peak at approximately 27.10 2θ, considered to be an anhydrate, was also found.

Compound 2 is predicted by MarvinSketch 24.1.0 to have PKa values of 1.4 and 6.4 for the two phosphate hydrogens, and should therefore form both mono- and di-salts with monobasic bases. A salt screen was carried out, resulting in the identification of 16 distinctive salts and their polymorphs, including two mono-sodium salts, two di-sodium salts, a mono-potassium salt, two erbumine salts, two betaine salts, an ethylenediamine salt, a mono-tromethamine salt, a mono-diethylamine salt, a mono-ammonia salt, a mono-deanol salt, two mono-L-lysine salts, and a hemi-magnesium salt (prepared by metathesis).

### Example 6: Thermodynamic Solubility

The thermodynamic solubility of compounds 1 to 4, genistein, and genistein-7-O-phosphate, were measured in fasted state simulated intestinal fluid (FaSSIF: 0.056% w/v lecithin, 0.161% w/v sodium taurocholate, 0.39% w/v potassium dihydrogen phosphate, and 0.77% w/v potassium chloride in deionized water). The compounds were weighed into the lower chambers of Whatman Mini-UniPrep vials, and 450 μL of FaSSIF was added to each vial to obtain an over-saturated suspension. The samples were vortexed for a minimum of 2 minutes and then placed on a shaker at room temperature at a speed of 800 rpm for 24 hours. After shaking, the samples were centrifuged at 25° C. for 20 minutes at 4000 rpm. Filtrates were prepared using the Whatman Mini-UniPrep Syringeless Filter Device (PTFE filter media with polypropylene housing, pore size 0.45 μm) from Cytiva, and dilutions were performed as required. Quantification of all samples was carried out using an LC-UV system. Analytes were separated using an Xbridge C18 column (4.6×100 mm, 3.5 μm), using mobile phases of 0.1% TFA in water (Phase A) and 0.1% TFA in acetonitrile (Phase B). The results are shown in Table 1 below.

TABLE 1
Thermodynamic solubility of genistein phosphate derivatives in FaSSIF

| Compound | Solubility (μM) | Solubility (μg/mL) |
|---|---|---|
| Genistein | 27 | 7.29 |
| Genistein-7-O-phosphate | 3573 | 1251 |
| Compound 1 | 12539 | 4768 |
| Compound 2 | 7815 | 2971 |
| Compound 3 | 10208 | 5005 |
| Compound 4 | 612 | 293 |

### Example 7: Stability of Genistein Phosphate Derivatives in Simulated Gastric Fluid

The stability of compounds 1 to 4, and the control compound omeprazole, were measured in simulated gastric fluid (SGF: 0.2% w/v sodium chloride, 0.7% v/v hydrochloric acid, and 0.32% w/v pepsin in deionized water, pH 1.2±0.05). Stock solutions of the compounds (10 mM) were diluted to 200 μM in DMSO, then duplicate 2 μL aliquots added to 96-well plates for time points 0, 1, 2, 6, and 24 hours. SGF (198 μL) was added to all wells except those for time 0, to give a starting compound concentration of 2 μM; and the samples were incubated at 37° C. with agitation at 600 rpm for the prescribed times. After incubation, 200 μL of each sample was extracted and mixed with 400 μL cold ACN containing 200 ng/mL tolbutamide and labetalol as internal standards. The samples were centrifuged at 4000 μm for 20 minutes at 4° C., and the supernatants (60 μL) each diluted with purified water (180 μL) for LC/MS/MS analysis. The percentage of the test compound remaining at each time point was calculated based on the ratio of the peak area of the analyte to the internal standards. The results are shown in Table 2 below.

TABLE 2
Stability of genistein phosphate derivatives in SGF

| | Percentage remaining at time: | | | | |
|---|---|---|---|---|---|
| Compound | 0 | 1 hour | 2 hours | 6 hours | 24 hours |
| Compound 1 | 100 | 94.4 | 91.5 | 83.8 | 42.1 |
| Compound 2 | 100 | 100 | 104 | 116 | 113 |
| Compound 3 | 100 | 95.1 | 101 | 96.1 | 51.4 |
| Compound 4 | 100 | 104 | 110 | 114 | 106 |
| Omeprazole | 100 | 35.4 | 23.1 | 4.2 | 0 |

### Example 8: Intestinal Stability of Genistein Phosphate Derivatives

The stability of compounds 1 and 2, and the control compound 7-hydroxycoumarin, were measured using human, rat, and mouse intestinal S9 fractions (Xenotech). Test compound working solutions were prepared by diluting 5 μL of 10 mM stock solutions in DMSO with 495 μL AN. S9 solutions were prepared by diluting the obtained S9 solutions to 1.0 mg/mL in 100 mM potassium phosphate buffer at pH 7.4 containing 10 mM D-saccharic acid-1,4-lactone. An Apricot automation workstation was used to add 100 μL/well of S9 solution to all reaction plates (Blank, T0, T5, T15, T30, T45, T60 and NCF60); 2 μL/well of compound working solutions were added to all 96-well reaction plates except the blank (T0, T5, T15, T30, T45, T60, and NCF60); 600 μL/well of stop solution (4° C. ACN containing 200 ng/mL tolbutamide and 200 ng/mL labetalol as internal standards) was added to the T0 reaction plate; all plates were pre-incubated at 37° C. for 10 minutes; a cofactor regenerating system (98 μL/well) was added, excluding the NCF60 plate which instead received 98 μL/well of 100 mM potassium phosphate buffer. All plates were then incubated at 37° C. To terminate reactions, 600 μL/well of stop solution was applied at specific end time points. After sealing and a 10-minute shaking, plates were centrifuged at 4000 rpm and 4° C. for 20 minutes. Concurrently, 300 μL/well of HPLC grade water was loaded onto eight new 96-well bioanalysis plates. Post-centrifugation, 100 μL supernatant from each reaction plate was transferred to its corresponding bioanalysis plate, shaken for 10 minutes, and then subjected to LC-MS/MS analysis (Shimadzu LC-40D LC system coupled to a Sciex Qtrap 6500 Plus mass spectrometer, using both positive and negative ion modes).

In human intestinal S9, compounds 1 and 2 had half-lives of 28.7 and 26.8 minutes and intrinsic clearance rate of 48.4 μL/min/mg and 51.7 μL/min/mg respectively; in mouse intestinal S9, the corresponding values were 29.7 and 68.1 minutes and 46.7 and 20.3 μL/min/mg respectively; and in rat intestinal S9, the corresponding values were 2.6 and 48.3 minutes and 526.2 and 28.7 μL/min/mg respectively. Considering normalized control factor remaining data, rat and human were closest with 24.9% (human) and 30.4% (rat) for compound 1, and 56.6% (human) and 68.6% (rat) for compound 2; making the rat a convenient animal for pharmacokinetic experiments. Compounds 1 and 2 showed a noticeable absence of formation of glucuronidation, sulfation, and glutathione conjugates, suggesting that these metabolic pathways might not significantly contribute to the metabolism of the compounds under the experimental conditions employed.

### Example 9: Pharmacokinetic Studies in the Rat

The pharmacokinetics of compounds 1 and 2, and the control compound and metabolite genistein, were tested in solution (all compounds at 2.5 mg/mL; compounds 1 and 2 in 20% polyethylene glycol 400, 10% Labrasol (caprylocaproyl polyoxyl-8 glycerides), 7% polysorbate 80, remainder water; genistein in 30% Labrasol, 7% polysorbate 80, remainder water) in the fasted male SD rat (3 rats/group) at nominal doses of 25 mg/Kg, and plasma concentrations of the compounds measured at 5, 10 and 20 minutes and 1, 2, 3, 4, 6 and 8 hours after dosing. Genistein showed a mean tₘₐₓ of 10 minutes, a mean t_{1/2} of around 1.3 hours, a mean cₘₐₓ of around 300 ng/mL, and a mean AUC₀₋ₗₐₛₜ of around 500 ng·h/mL; compounds 1 and 2 both showed mean tₘₐₓ of around 8 minutes and a mean cₘₐₓ of around 15 ng/mL (compound 1) and 30 ng/mL (compound 2), with a very rapid decline in concentration. For both compounds 1 and 2, the genistein metabolite showed a mean tₘₐₓ of around 15 minutes, with a mean t_{1/2} of around 3 hours (compound 1) and over 10 hours (compound 2), and a mean AUC₀₋ₗₐₛₜ of around 160 ng·h/mL (compound 1) and around 200 ng·h/mL (compound 2). The pharmacokinetics of compound 2, and the control compound and metabolite genistein, were tested in solid form (powder in mini-capsules) at 100 mg/Kg for compound 2 (alone or together with 20 mg/Kg inulin) and 71 mg/Kg for genistein (alone or together with 14.2 mg/Kg inulin, in each case to give equimolar dosing with compound 2) in the fasted male SD rat (3 rats/group), and plasma concentrations of the compounds measured at 5, 10 and 20 minutes and 1, 2, 3, 4, 6 and 8 hours after dosing. Genistein showed a mean tₘₐₓ of around 3 hours, a mean t_{1/2} of around 2 hours, a mean cₘₐₓ of around 160 ng/mL, and a mean AUC₀₋ₗₐₛₜ of around 500 ng·h/mL (with considerable variability), while addition of inulin steepened the AUC curve, delaying (to around 5 hours) and slightly reducing the peak, and significantly reducing the mean AUC₀₋ₗₐₛₜ to around 300 ng·h/mL. Compound 2 showed a mean tₘₐₓ of around 10 minutes (with considerable variability) and mean t_{1/2} of around 2 hours, with a slower rapid decline in concentration than for solution dosing; addition of inulin had little effect on the AUC curve. For compound 2, the genistein metabolite showed a mean tₘₐₓ of around 20 minutes and a similar shape of AUC curve to that seen for solution dosing; again, addition of inulin had little effect on the AUC curve.

Genistein has a log P of 3.04, while compound 2 has a log P of 1.59. The results of the solubility studies, the stability studies, and the pharmacokinetic studies demonstrate that the genistein phosphate derivatives of this invention have better solubility and better drug delivery, including higher Cₘₐₓ, than genistein itself, while being highly effective prodrugs for genistein. Accordingly, the genistein phosphate derivatives of this invention will provide suitable treatments for conditions treatable by genistein or a genistein derivative, such as dilated cardiomyopathy (DCM)—including LMNA- and FLNC-associated DCM, Duchenne muscular dystrophy, heart failure, inflammatory bowel disease/ulcerative colitis/Crohn's disease, obesity and obesity-induced diabetes, osteoarthritis, pancreatitis, rheumatoid arthritis, and transthyretin amyloid cardiomyopathy, and indeed are expected to be better than genistein for these conditions; and will also provide suitable treatments for Down syndrome.

### Example 10: Dilated Cardiomyopathy and Heart Failure

Cardiomyopathies are diseases of the heart muscle that render the heart unable to properly pump enough blood to the body. In the dilated form of cardiomyopathy (called dilated cardiomyopathy or DCM), the heart is enlarged. As the heart enlarges, it becomes less effective in pumping blood, which then leads to symptoms of heart failure and irregular heart rhythms (arrhythmias). DCM is principally characterized by left ventricular enlargement and/or a reduction in systolic function, more precisely described as a reduction in left ventricular ejection fraction less than <40% or fractional shortening less than 25% (Hershberger et al., “Clinical and genetic issues in dilated cardiomyopathy: a review for genetics professionals”, Genet. Med., 12, 655-667 (2010)). It is estimated that approximately 1 out of every 2500 persons has DCM, although the disease is probably even more common. DCM affects both men and women and can affect both adults and children. As with other types of cardiomyopathies, DCM is a chronic disease without a known cure. However, the treatments currently available can significantly improve its course. There are many possible causes of dilatation and dysfunction of the heart, such as coronary artery disease, infection, and excessive use of alcohol. In cases where the cause of DCM is unknown, the condition is called “idiopathic” dilated cardiomyopathy. About one-third to one-half of patients with idiopathic DCM have a family history of the disease in one or more relatives. These patients are considered to have familial dilated cardiomyopathy. Familial DCM is caused by defective genes that affect the function of the heart muscle. Several familial DCM genes are currently known, whereas others are still under investigation.

DCM may involve mutations in one or more of the following genes: LMNA, FLNC, BAG3, DES, MYH7, PLN, RBM20, SCN5A, TNNCI, TNNT2, TTN, DSP, ACTC1, ACTN2, JPH2, NEXN, TNNI3, TPM1, VCL, ABCC9, ANKRDI, CSRP3, CTFI, DSG2, DTNA, EYA4, GATAD1, ILK, LAMA4, LDB3, MYBPC3, MYH6, MYL2, MYPN, NEBL, NKX2-5, OBSCN, PLEKHM2, PRDM16, PSEN2, SGCD, TBX20, TCAP, TNNI3K, LRRC10, NPPA, MIB1, MYL3, PDLIM3, PKP2, PSEN1, and others. These genes can contribute to familial DCM, which can result in abnormally elevated platelet-derived growth factors (PDGF). In idiopathic DCM, and particularly in familial DCM, a pathogenic variant of the lamin A (LMNA) or filamin C (FLNC) gene may be the underlying cause for DCM (Hershberger et al.; Brayson et al., “Current insights into LMNA cardiomyopathies: Existing models and missing LINCs”, Nucleus, 8, 17-33 (2017). LMNA is a gene that encodes the intermediate filament proteins, lamin A and C, which localize between the nuclear membrane and the chromatin. Mutations in the LMNA gene lead to disruption of cellular functions, which can result in a milieu of diseases referred to as laminopathies (Brayson et al.; Lu et al., “LMNA cardiomyopathy: cell biology and genetics meet clinical medicine”, Dis. Model Mech., 4, 562-568 (2011)). Laminopathies all share a degree of nuclear fragility, altered nuclear architecture, impaired nuclear signaling and transcriptional activation through alterations in adaptive or protective mechanisms. LMNA is one of few established genes that has a clear genotype to clinical phenotype relationship, which has proven to be associated with conduction defects, malignant ventricular arrhythmias, and supraventricular arrhythmias preceding the development of left ventricular dilation and heart failure DCM (Hershberger et al.). Unlike other cases of familial DCM, often the first sign of LMNA-related DCM is sudden cardiac arrest leading to death, owing to prevalence of arrhythmias, e.g., ventricular tachycardia and/or ventricular fibrillation (Lee et al., “Activation of PDGF pathway links LMNA mutation to dilated cardiomyopathy”, Nature, 572, 335-340 (2019)). Specific modifications of lamin A that have been associated with DCM include Q6*, R26G, K32del, A57P, L59R, R60G, E82K, L85R, K117fs, R133P, L140R, E145K, E161K, N195K, E203G, H222P, R225Q, R249Q, Y267C, R298C, D300G, E358K, M371K, R377H, R453W, R527P, T528R, L530P, R541G, R571S, S573L, V607V, G608S, G608G, and Q656Q.

Filamin C, encoded by the FLNC gene, is an isoform of the filamin family, predominantly expressed in skeletal and cardiac muscle (Razinia et al., “Filamins in mechanosensing and signaling”, Ann. Rev. Biophys., 41, 227-246 (2012); Zhou et al., “Filamins in cell signaling, transcription and organ development”, Trends Cell Biol., 20, 113-123 (2010)). Filamin C modulates cell stiffness by regulating cross-linking of actin filaments and anchorage of the actin-cytoskeleton to transmembrane proteins at cell-cell adhesion sites. Because of its interactions with transmembrane and Z-disc proteins, mutations in filamin C would be expected to dysregulate intracellular signaling. Mutations in filamin C have been associated with myofibrillar skeletal myopathies, hypertrophic cardiomyopathy, restrictive cardiomyopathy, DCM, and arrhythmogenic cardiomyopathy (Brodehl et al., “Mutations in FLNC are Associated with Familial Restrictive Cardiomyopathy”, Hum. Mutat., 37, 269-279 (2016); Tucker et al., “Novel Mutation in FLNC (Filamin C) Causes Familial Restrictive Cardiomyopathy”, Circ. Cardiovasc. Genet., 10 (2017); Valdes-Mas et al., “Mutations in filamin C cause a new form of familial hypertrophic cardiomyopathy”. Nat. Commun., 5, 5326 (2014)). Carriers of filamin C truncating mutations are at high risk of developing dilated and arrhythmogenic cardiomyopathies (Begay et al., “FLNC Gene Splice Mutations Cause Dilated Cardiomyopathy”, JACC Basic Transl. Sci., 1, 344-359 (2016); Begay et al., “Filamin C Truncation Mutations Are Associated With Arrhythmogenic Dilated Cardiomyopathy and Changes in the Cell-Cell Adhesion Structures”, JACC Clin. Electrophysiol., 4, 504-514 (2018)). Specific modifications of filamin C that have been associated with DCM include Y83*, F106L, R991*, P963R, A1183L, A1186V, V1198Gfs*64, S1642L, G1891Vfs61X, G2011E, 12160F, E2189X, V2297M, P2298L, G2299S, R2318W, R2410C, and Y2563C.

The LMNA^{H222P/H222P} mouse is a well-established model for studying dilated cardiomyopathy and heart failure, with cardiac dysfunction typically beginning to manifest at around 16 weeks of age. In this study, 16-week-old LMNA^{H222P/H222P} mice underwent baseline echocardiography prior to treatment. Compound 2 was then administered continuously for 4 weeks using osmotic pumps, with DMSO as the vehicle control. At the end of the treatment period, echocardiography was performed again to assess the impact of compound 2 on cardiac function compared to the control group. Treatment with compound 2 reduced the decrease in ejection fraction and fractional shortening seen in the control group, and reduced left ventricular volume and diameter where increases were seen in the control group. In a FLNC knockout model using the Myh6-Cre:FLNC^{wt/F} mouse, 16-week-old mice underwent baseline echocardiography, were treated with compound 2 at 50 mg/Kg/day or vehicle by gavage for 6 weeks, and underwent echocardiography to assess the change in cardiac function. Treatment with compound 2 statistically significantly improved contractility (fractional shortening) from baseline; and reduced left ventricular end-diastolic diameter and end-systolic diameter and improved contractility over vehicle-treated animals. The genistein phosphate derivatives of this invention are therefore expected to be useful for the treatment of subjects with dilated cardiomyopathy.

In addition, Green et al., “Genistein, a natural product from soy, is a potent inhibitor of transthyretin amyloidosis”, Proc. Nat'l Acad. Sci., 102(41), 14545-14550 (2005) have shown that genistein is an excellent inhibitor of transthyretin tetramer (TTR) dissociation and amyloidogenesis, reducing acid-mediated fibril formation to <10% of that exhibited by TTR alone; it also inhibits the amyloidogenesis of the most common familial amyloid polyneuropathy and familial amyloid cardiomyopathy mutations in TTR: V30M and V122I, respectively; it additionally inhibits tetramer dissociation under physiological conditions thought to lead to slow amyloidogenesis in humans; and that it exhibits highly selective binding to TTR in plasma over all of the other plasma proteins. Matori et al., “Genistein, a Soy Phytoestrogen, Reverses Severe Pulmonary Hypertension and Prevents Right Heart Failure in Rats”, Hypertension, 60(2), 425-430 (2012), state that pretreatment with genistein has been shown to attenuate the development of pulmonary hypertension (PH), and demonstrated in a rat model that genistein also reversed monocrotaline-induced preexisting PH, reducing right ventricular pressure and restoring right ventricular ejection fraction, and prevented the progression of severe PH to right heart failure. Jafari et al., “Pharmacological Effects of Genistein on Cardiovascular Diseases”, Evid.-Based Complement. Alternat. Med., 2023, U.S. Pat. No. 8,250,219 (2023), https://doi.org/10.1155/2023/8250219, give a literature review on studies of genistein in cardiovascular diseases, saying that “The results of the studies showed that genistein intake has a promising effect on improving cardiac dysfunction, ischemia, and reperfusion of the heart, decreasing cardiac toxicity, modulating lipid profile, and lowering blood pressure. The preventive effects of genistein on experimental models of studies were shown through mechanisms such as anti-inflammatory, antioxidant, and immunomodulatory effects. Pharmacological effects of genistein on cardiac dysfunction, cardiac toxicity, lipid profile, and hypertension indicate the possible remedy effect of this agent in the treatment of CVD.” As prodrugs of genistein, and considering the anti-inflammatory effects of the genistein phosphate derivatives of this invention, these genistein phosphate derivatives are therefore expected also to be useful for the treatment of subjects with cardiovascular conditions such as transthyretin amyloid cardiomyopathy (ATTR-CM) and heart failure, including heart failure with reduced ejection fraction.

### Example 11: Duchenne Muscular Dystrophy

According to Messina et al., “The soy isoflavone genistein blunts nuclear factor kappa-B, MAPKs and TNF-α activation and ameliorates muscle function and morphology in mdx mice”, Neuromuscul. Dis., 21, 579-589 (2011), “Duchenne muscular dystrophy (DMD) is the most common lethal X-linked recessive disorder, affecting 1 in 3500 live male births. DMD children show early symptoms of muscle degeneration, frequently develop contractures, and lose the ability to walk by 13 years of age. With disease. progression, most patients succumb to death from respiratory failure and cardiac dysfunction in early adulthood. The primary cause of this disease stems from the lack of the protein dystrophin, which is essential for the structural and functional integrity of muscle membrane. Dystrophin absence results in membrane damage, allowing massive infiltration of immune cells, chronic inflammation, necrosis, and severe muscle degeneration . . . . The mdx mouse, a genetically homologous DMD model, is frequently used to study the disease pathogenesis, despite relevant clinical and pathological differences.” Messina et al. say that: “Five-week old mdx mice received for five weeks: genistein (daily or 3-times/week), methylprednisolone or vehicle. Genistein treatment: (1) increased forelimb strength and strength normalized to weight; (2) reduced serum creatine-kinase levels; (3) reduced markers of oxidative stress; (4) reduced muscle necrosis and enhanced regeneration. The positive results were more evident with the daily administration of genistein and were comparable to the effect of corticosteroids.” Four-month-old mdx mice were treated with compound 2 at 20 mg/Kg/day by gavage, and blood analyzed for cytokines. Leptin was shown to be significantly reduced, and a number of cytokines, including BAFF, BTC, CCL7, CCL11, CSF3, CXCL10, IL-1α, IL-1β, IL-5, IL-10, IL-18, IL-19, IL-2RA, IL-7RA, LIF, MIP-10, MIP2, TNF-α, and VEGF, appeared to be reduced. The genistein phosphate derivatives of this invention are therefore expected to be useful for the treatment of subjects with Duchenne muscular dystrophy.

### Example 12: Inflammatory Bowel Disease, Ulcerative Colitis, and Crohn's Disease

Genistein has been shown to treat induced colitis in rodent models (see, for example, Alharbi et al., “Therapeutic effects of genistein in experimentally induced ulcerative colitis in rats affecting mitochondrial biogenesis”, Mol. Cell Biochem., 479, 431-444 (2024), ulcerative colitis (UC) induced with intracolonic administration of 4% acetic acid, treatment with 25 mg/Kg/day genistein; Ha et al., “Genistein alleviates dextran sulfate sodium-induced colitis in mice through modulation of intestinal microbiota and macrophage polarization”, Eur. J. Nutr., https://doi.org/10.1007/s00394-024-03391-1, UC induced with dextran sulfate sodium (DSS), treatment with 20 or 40 mg/Kg/day genistein). Mice, placed on a soy-free diet for 14 days, were administered 3.5% DSS in their drinking water for 8 days to induce colitis, and either untreated or treated with compound 2 at 50 mg/Kg daily by gavage from day 1 (prevention) or from day 5 (intervention); and monitored for clinical indicators of colitis, including stool consistency, rectal bleeding, and changes in body weight. As expected, DSS treatment alone resulted in significant weight loss, diarrhea, and colon shortening, which are hallmark indicators of colitis severity in this model. Administration of compound 2 significantly ameliorated these symptoms: treated mice displayed reduced weight loss, rectal bleeding, and diarrhea, and showed improved colon length and histological improvement of the colon, in both prevention and intervention modes. The genistein phosphate derivatives of this invention are therefore expected to be useful for the treatment of subjects with inflammatory bowel disease, ulcerative colitis, and Crohn's disease.

### Example 13: Obesity and Obesity-Induced Diabetes

Genistein reduced fasting blood glucose by ˜30% in wild-type mice fed a high fat diet for 12 weeks followed by intraperitoneal injection of 200 mg/Kg genistein in corn oil for 14 days. Genistein also reduced fasting blood glucose by ˜57% and HbA1C by ˜37% in Db/Db mice fed a high fat diet for 6 weeks followed by oral gavage of 50 mg/Kg genistein in corn oil for 47 days. Three groups of 16 month-old mice were implanted with 7-day osmotic pumps dispensing vehicle, 0.24 mg/Kg/day, and 2.4 mg/Kg/day of compound 2. All three groups of animals lost ˜1% of body weight over the 7 days. Both treatment groups showed lowered fasting blood glucose relative to the control group, with the 2.4 mg/Kg/day group showing a significant decrease. Leptin and IL-13 blood levels were also reduced in a dose-dependent manner, with the 2.4 mg/Kg/day group showing levels similar to those of 6 month-old mice, suggesting healthier adipose tissue. The genistein phosphate derivatives of this invention are therefore expected to be useful for the treatment of subjects with obesity and obesity-induced diabetes.

### Example 14: Osteoarthritis

In the mini-joint model, human mesenchymal stem cells (MSCs) were differentiated into chondrocytes in pellet culture over a period of 3 weeks. The chondrocyte pellets were then maintained in a medium containing 0.5 ng/mL of TGF-β for 1 week. Inflammation was induced by treating the pellets with 1 ng/mL of IL-1β for 1 day. Following this, the pellets were treated with compound 2 at up to 5 μM together with 0.01 ng/mL of IL-1β to sustain the inflammatory environment for 3 days. At the conclusion of the treatment, the pellets were collected for PCR analysis. Compound 2 has been shown to promote chondrogenesis (expression of collagen A) in this model, increasing COL2, and decreasing MMP12 and TNF-α.

### Example 15: Pancreatitis

According to Ashraf et al., “A Clinical Overview of Acute and Chronic Pancreatitis: The Medical and Surgical Management”, Cureus, 13(11): e19764 (2021), “An inflammatory process involving the pancreas, known as pancreatitis, can be categorized as either acute or chronic and may present in one of many ways. The clinical manifestations of acute pancreatitis are generally limited to epigastric or right upper quadrant pain, while manifestations of chronic pancreatitis are broader and may include abdominal pain in tandem with signs and symptoms of pancreatic endocrine and exocrine insufficiency. An understanding of the initial insult, proper classification, and prognosis are all factors that are of paramount importance as it pertains to managing patients who are afflicted with this disease.” Chronic pancreatitis is a progressive inflammatory and fibrotic disease, inflicting irreversible damage to the pancreas and subsequently impeding its dual exocrine and endocrine roles. Recurrent episodes of pancreatitis not only intensify this damage but also elevate the associated morbidity and mortality rates. Pancreatic inflammatory diseases, encompassing acute pancreatitis (AP), RAP and CP, lead to significant clinical and economic burdens. They rank prominently as causes for gastrointestinal disorder-related hospitalizations, presenting with debilitating pain often necessitating narcotic interventions and hospital stays. Additionally, complications such as pseudocysts, and both exocrine and endocrine pancreatic insufficiencies, further complicate the clinical picture continuum linking pancreatitis and pancreatic cancer. Statistics reveal that individuals with AP face a 21% risk of recurrence, with nearly 36% of such recurrent cases advancing to CP. This progression from recurrent AP episodes subsequently heightens the risk of pancreatic cancer, escalating it by a factor of 13.3. External risk factors, beyond alcohol and tobacco consumption, include diabetes and gallstones. As described by Siriviriyakul et al., “Genistein attenuated oxidative stress, inflammation, and apoptosis in L-arginine induced acute pancreatitis in mice”, BMC Complement. Med. Ther., 22:208 (2022), genistein, at both low (10 mg/Kg/day) and high (100 mg/Kg/day) doses, ameliorated the pancreatitis pathology by significantly reducing edema, necrosis and cell death induced by L-arginine, and also significantly reduced serum amylase, IL-6 and CRP levels. Additionally, it exhibited anti-inflammatory actions by curbing pro-inflammatory molecules like NF-κB and 4-HNE expression. Xia et al., “Genistein protects against acute pancreatitis via activation of an apoptotic pathway mediated through endoplasmic reticulum stress in rats”, Biochem. Biophys. Res. Commun., 509(2), 421-428 (2019), further corroborated these findings, showcasing the benefits of genistein in two distinct acute pancreatitis models. Acute edematous pancreatitis (AEP) was induced by administering cerulein through 2-hour intervals. On the other hand, acute necrotizing pancreatitis (ANP) was induced by introducing 3.5% sodium taurocholate (0.1 mL/100 g) into the biliopancreatic duct. In the cerulein group (AEP), the pancreas displayed mild edema and infiltration of inflammatory cells, with no significant parenchymal necrosis or hemorrhage. In contrast, ANP animal models exhibited extensive parenchymal necrosis and hemorrhage in the pancreas. Treatment with genistein mitigated necrosis, edema and inflammatory infiltration compared to the experimental models.

Using the cerulein model, acute edematous pancreatitis was induced in two test groups of mice by eight hourly intraperitoneal injections of 50 μg/Kg cerulein, with control group mice receiving injections of physiological saline. Compound 2, at 100 mg/Kg, was administered to one of the cerulein groups by gavage 30 minutes after the first and fourth cerulein injections. Mice were sacrificed one hour after the last injection and tissues were collected for histological analysis. Initial findings suggest that compound 2 is efficacious in reversing the acute edematous pancreatitis marked by necrosis, inflammation and edema in the pancreas. Recurrent acute pancreatitis (RAP) and chronic pancreatitis (CP) were induced by repetitive cerulein injections every third day over a span of 14 days (RAP) or 6 weeks (CP). Compound 2 was administered daily at 50 mg/Kg on the last 10 consecutive days for RAP and at 100 mg/Kg on the last 4 weeks for CP, with vehicle controls. Mice were sacrificed 1 day after the last cerulein injection and tissues were collected for histological analysis. In the CP model, the cerulein injections induced extensive acinar cell damage. Mice treated with compound 2 exhibited marked improvement over untreated mice, including significant reduction of acinar cell death and increase in acinar cell density in the treated group compared to the untreated group, underscoring the potential of compound 2 in preserving pancreatic structure and function. Based on the effects of genistein and this Example, the genistein phosphate derivatives of this invention are therefore expected to be useful for the treatment of subjects with pancreatitis.

### Example 16: Pancreatic Ductal Adenocarcinoma

Chronic pancreatitis (CP) is a significant risk factor for pancreatic cancer due to persistent inflammation in the pancreas, which can lead to DNA damage and mutations in pancreatic cells. A meta-analysis has shown that when compared with controls, there is 13-fold greater lifetime risk of pancreatic cancer in CP. The chronic inflammatory state in CP fosters an environment conducive to the development of pancreatic ductal adenocarcinoma (PDAC), the most common type of pancreatic cancer; and the fibrotic and scarred tissue in CP also contributes to changes in the pancreatic microenvironment, further promoting tumorigenesis. Based on the effect of genistein and compound 2 as seen in Example 15, the genistein phosphate derivatives of this invention are therefore expected to be useful for the treatment of subjects with pancreatic ductal adenocarcinoma.

### Example 17: Systemic Vasculitis

Systemic vasculitis is a group of disorders characterized by inflammation of the blood vessels, which can affect arteries, veins, and capillaries throughout the body. This inflammatory process leads to endothelial cell activation, immune cell infiltration, and the release of pro-inflammatory cytokines, which can compromise vascular integrity and lead to tissue and organ damage. Systemic vasculitis is associated with a complex pathophysiology involving immune-mediated injury and dysregulated inflammatory signalling, including increased activity in the NF-κB and MAPK pathways, upregulation of inflammatory cytokines such as TNF-α, IL-1β, and IL-6, and oxidative stress. Genistein is known to inhibit key inflammatory pathways, has demonstrated efficacy in reducing inflammation in models of rheumatoid arthritis and colitis, both of which share underlying inflammatory mechanisms with systemic vasculitis, and has been shown to alleviate vasculitis in animal models (see, for example, Jia et al., “Genistein inhibits TNF-α-induced endothelial inflammation through the protein kinase pathway A and improves vascular inflammation in C57BL/6 mice”, Int. J. Cardiol., 168(3), 2637-2645 (2013); and Xie et al., “Genistein alleviates chronic vascular inflammatory response via the miR-21/NF-κB p65 axis in lipopolysaccharide-treated mice”, Mol. Med. Rep., 23, 192 (2021)). Based on the observed anti-inflammatory efficacy of compound 2 in models of acute and chronic pancreatitis seen in Example 15, the genistein phosphate derivatives of this invention are therefore expected to be useful for the treatment of subjects with inflammatory diseases including systemic vasculitis.

### Example 18: Down Syndrome

Individuals with Down Syndrome (DS), a genetic condition caused by the trisomy of chromosome 21, exhibit a distinct immunological profile marked by a chronic state of hyperinflammation. This is primarily driven by the overexpression of immune-regulatory genes located on chromosome 21, notably IFN receptor genes such as IFNAR1/2, IFNGR2, and IL10RB. These genes are key components of the type I interferon (IFN) signaling pathway, which plays a central role in orchestrating antiviral responses and immune regulation. Their overexpression in DS leads to heightened sensitivity to interferon signaling, promoting a sustained pro-inflammatory environment. This dysregulated immune signaling results in the overproduction of pro-inflammatory cytokines, including interleukin-6 (IL-6) and tumor necrosis factor-alpha (TNF-α), as well as persistent immune activation. Elevated levels of oxidative stress further exacerbate this inflammatory state. The chronic immune activation seen in DS contributes to a higher prevalence of autoimmune diseases, such as autoimmune thyroiditis and celiac disease, and increases susceptibility to infections, particularly respiratory and gastrointestinal infections. Interestingly, while DS individuals experience a heightened innate immune response, their adaptive immune system is paradoxically impaired. Studies have documented reductions in T and B cell numbers, along with defects in their functionality. This impaired adaptive immunity leaves DS individuals vulnerable to recurrent infections and contributes to a suboptimal response to vaccinations. These immune dysfunctions play a critical role in DS's increased risk of hematological malignancies, including acute megakaryoblastic leukemia (AMKL), which is disproportionately higher in this population. A growing body of research has identified the Janus kinase (JAK)-signal transducer and activator of transcription (STAT) pathway as a key driver of immune dysregulation in DS. The JAK-STAT pathway is crucial for transmitting signals from cytokines and interferons to the cell nucleus, leading to the expression of inflammatory genes. Overactivation of this pathway in DS has been implicated in the persistent inflammatory state and contributes to the development of immune-related comorbidities. The chronic inflammatory state in DS has far-reaching consequences beyond immune dysfunction. Inflammatory processes contribute to the development of neurodegenerative conditions, such as early-onset Alzheimer's disease, which is prevalent in individuals with DS due to the overexpression of APP (amyloid precursor protein), another gene on chromosome 21. Oxidative stress and chronic inflammation are thought to accelerate amyloid plaque formation and neurodegeneration in DS, making anti-inflammatory interventions a potential avenue for mitigating cognitive decline. Furthermore, metabolic disorders, including obesity and insulin resistance, are increasingly recognized in the DS population. Chronic inflammation is a known contributor to metabolic dysregulation, and the immune-metabolic crosstalk in DS may be a key factor in the development of these conditions.

Waugh et al., “Triplication of the interferon receptor locus contributes to hallmarks of Down syndrome in a mouse model”, Nat. Genet., 55, 1034-1047 (2023), note that the B6.129S7-Dp(16Lipi-Zbtb21)1Yey/J mouse model of DS, sometimes referred to as the Dp16 mouse, carries a segmental duplication of mouse chromosome 16 causing triplication of ˜120 protein-coding genes orthologous to those on human chromosome 21, including the Ifnr cluster; and that Dp16 mice display key phenotypes of DS including hyperactive IFN signaling, a dysregulated antiviral response, increased prevalence of heart defects, developmental delays, cognitive impairments and craniofacial anomalies. The TLR3 agonist polyinosinic-polycytidylic acid, poly(I:C), is an innate immune stimulus known to trigger an IFN response. Three groups of Dp16 mice were dosed from day ˜7 through 10 by gavage at 10 mg/Kg, twice/day for the first 5 days and once/day thereafter, with vehicle (2 groups) or compound 2 in vehicle (1 group), and one of the vehicle groups and the compound 2 group were dosed by injection with poly(I:C) on day 7, while the other vehicle group was given a sham injection. Treatment with compound 2 ameliorated the increased expressions of cytokines, IFNα and -7, TNF-α, IL-2, -5, -6, -10, and -12P70, and attenuated the inflammatory response, including ameliorating the skin thickening, induced by Poly(I:C) in these Dp16 mice. Transcriptome analysis of liver samples identified gene sets substantially dysregulated in the Dp16 mice and attenuated by treatment with compound 2; including signatures of increased cell proliferation (E2F targets and G2/M checkpoint), increased JAK/STAT and interferon signaling, and decreased fatty acid metabolism. The genistein phosphate derivatives of this invention are therefore expected to be useful for the treatment of subjects with Down syndrome.

### Example 19: Rheumatoid Arthritis

Based on the studies in genistein, and the studies of compound 2 described here, the genistein phosphate derivatives of this invention are expected to exert anti-inflammatory and disease-modifying effects by inhibiting the activity of the MAPK/NF-κB signaling pathway. This pathway plays a crucial role in the inflammatory processes and cellular responses that drive the pathological development of various musculoskeletal conditions, including rheumatoid arthritis, osteoarthritis, osteoporosis, and intervertebral disc degeneration. By inhibiting the MAPK/NF-κB signaling cascade, these compounds are expected to reduce the expression of key pro-inflammatory cytokines, such as TNF-α, IL-6, and IL-1β, which are central to the pathogenesis of rheumatoid arthritis. Additionally, their inhibition of these pathways may help to decrease synovial hyperplasia and joint destruction, thereby mitigating disease progression. Furthermore, their ability to modulate these signaling pathways suggests potential broader applications in related conditions involving inflammation and tissue degeneration.

Approximately 120 patients, ages 30-65, diagnosed with rheumatoid arthritis according to the 2010 ACR/EULAR classification criteria, are enrolled in a randomized, double-blind, placebo-controlled study to evaluate the efficacy of compound 2 in patients with moderate to severe rheumatoid arthritis. Participants are randomized to receive either oral compound 2 (500 mg capsule) or placebo once daily for a period of 24 weeks. The primary efficacy endpoint is the improvement in the Disease Activity Score-28 (DAS28) with C-reactive protein (CRP) at week 24. A clinically meaningful reduction in DAS28 score is defined as a reduction of ≥1.2 points from baseline. Secondary endpoints include: Improvement in physical function as measured by the Health Assessment Questionnaire Disability Index (HAQ-DI), reduction in morning stiffness and joint pain, and changes in levels of inflammatory cytokines such as TNF-α, IL-6, and IL-1β. It is predicted that patients receiving compound 2 will demonstrate a statistically significant improvement in DAS28 scores compared to the placebo group. Additionally, compound 2 is expected to reduce serum levels of pro-inflammatory cytokines and markers of joint damage such as matrix metalloproteinases (MMPs), thus providing evidence of its disease-modifying effects.

While this invention has been described in conjunction with specific embodiments and examples, it will be apparent to a person of ordinary skill in the art, having regard to that skill and this disclosure, that equivalents of the specifically disclosed materials and methods will also be applicable to this invention; and such equivalents are intended to be included within the following claims.

1. A compound of formula I or II: 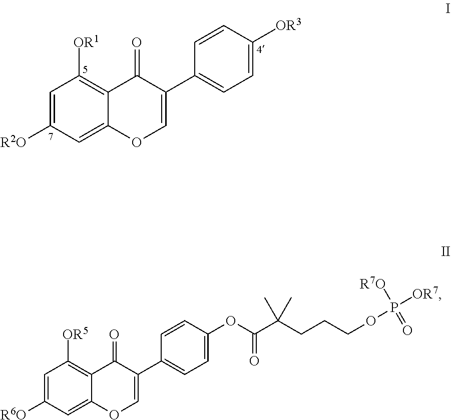
or a pharmaceutically acceptable salt thereof, where:
in formula I:
each of R¹, R², and R³ independently is selected from the group consisting of H, C₁₋₆ alkyl, and (R⁴O)₂P(O)CH₂—, provided that at least one of R¹, R², and R³ is (R⁴O)₂P(O)CH₂—; and
each R⁴ independently is selected from the group consisting of H and C₁₋₆ alkyl; and
in formula II:
each of R⁵ and R⁶ independently is selected from the group consisting of H, C₁₋₆ alkyl, and (R⁷O)₂P(O)CH₂—, and each R⁷ independently is selected from the group consisting of H and C₁₋₆ alkyl.

2. The compound of claim 1 that is a compound of formula I or a pharmaceutically acceptable salt thereof.

3. The compound of claim 2 where R¹ is H, or a pharmaceutically acceptable salt thereof.

4. The compound of claim 2 where each R⁴ is H, or a pharmaceutically acceptable salt thereof.

5. The compound of claim 2 that is (4-(5,7-dihydroxy-4-oxo-4H-chromen-3-yl)phenoxy)methyl dihydrogen phosphate, ((5-hydroxy-3-(4-hydroxyphenyl)-4-oxo-4H-chromen-7-yl)oxy)methyl dihydrogen phosphate, or ((5-hydroxy-4-oxo-3-(4-((phosphonooxy)methoxy)phenyl)-4H-chromen-7-yl)oxy)methyl dihydrogen phosphate, or a pharmaceutically acceptable salt thereof.

6. The compound of claim 5 that is ((5-hydroxy-3-(4-hydroxyphenyl)-4-oxo-4H-chromen-7-yl)oxy)methyl dihydrogen phosphate or a pharmaceutically acceptable salt thereof.

7. The compound of claim 6 that is ((5-hydroxy-3-(4-hydroxyphenyl)-4-oxo-4H-chromen-7-yl)oxy)methyl dihydrogen phosphate.

8. The compound of claim 1 that is a compound of formula II or a pharmaceutically acceptable salt thereof.

9. The compound of claim 8 where R⁵ and R⁶ are H, or a pharmaceutically acceptable salt thereof.

10. The compound of claim 8 where each R⁷ is H, or a pharmaceutically acceptable salt thereof.

11. The compound of claim 8 that is 4-(5,7-dihydroxy-4-oxo-4H-chromen-3-yl)phenyl 2,2-dimethyl-5-(phosphonooxy)pentanoate or a pharmaceutically acceptable salt thereof.

12. A pharmaceutical formulation comprising a therapeutically effective amount of a compound of claim 1 and a pharmaceutically acceptable excipient.

13. The pharmaceutical formulation of claim 12 that is an oral formulation.

14. The pharmaceutical formulation of claim 13 that is a tablet, a capsule, an oral solution, or an oral suspension.

15. The pharmaceutical formulation of claim 12 where the compound is ((5-hydroxy-3-(4-hydroxyphenyl)-4-oxo-4H-chromen-7-yl)oxy)methyl dihydrogen phosphate or a pharmaceutically acceptable salt thereof.

16. A method of treating a condition treatable by genistein or a genistein derivative in a subject, comprising administration to the subject of a therapeutically effective amount of a compound of claim 1.

17. A method of treating a condition selected from dilated cardiomyopathy, Duchenne muscular dystrophy, heart failure, inflammatory bowel disease/ulcerative colitis/Crohn's disease, obesity and obesity-induced diabetes, osteoarthritis, pancreatitis, pancreatic ductal adenocarcinoma, rheumatoid arthritis, systemic vasculitis, and transthyretin amyloid cardiomyopathy, and Down syndrome, in a subject, comprising administration to the subject of a therapeutically effective amount of a compound of claim 1.

18. The method of claim 16 where the compound is ((5-hydroxy-3-(4-hydroxyphenyl)-4-oxo-4H-chromen-7-yl)oxy)methyl dihydrogen phosphate or a pharmaceutically acceptable salt thereof.

19. The method of claim 16 where the dose of the compound is 0.1-100, 0.2-80, 0.3-60, 0.4-40, or 0.5-20 mg/Kg/day.

20. The method of claim 16 where the dose of the compound is 1-2,500, 2-2,000, 5-1,500, 10-1,000, 15-750, or 20-500 mg twice/day.
